(19) United States
(12) Patent Application Publication
(10) Pub. No.: US 2021/0298624 A1
(43) Pub. Date: Sep. 30, 2021

(54) NEUROANATOMICAL TRACT VISUALIZATION USING SYNAPTIC CONNECTIVITY GRAPHS

(71) Applicant: X Development LLC, Mountain View, CA (US)

(72) Inventors: Sarah Ann Laszlo, Mountain View, CA (US); Jeffrey Bush, Los Altos, CA (US); Bin Ni, Fremont, CA (US); Matthew Sibigtroth, Richmond, CA (US)

(21) Appl. No.: 16/829,179

(22) Filed: Mar. 25, 2020

(51) Int. Cl. A61B 5/04; A61B 5/00

(52) U.S. Cl. CPC A61B 5/04001; A61B 5/742; A61B 5/0042

(57) ABSTRACT

Methods, systems, and apparatus, including computer programs encoded on a computer storage medium, for neuroanatomical tract visualization using synaptic connectivity graphs. In one aspect, a method comprises: presenting, to a user and through a display, a representation of a synaptic connectivity graph representing synaptic connectivity between neurons in a brain of a biological organism; receiving, from the user, data specifying a seed neuron in the brain; identifying a neuroanatomical tract corresponding to the seed neuron in the brain; and presenting, to the user and through the display, a geometric representation of at least a portion of the brain of the biological organism that visually distinguishes the neuroanatomical tract corresponding to the seed neuron at neuronal resolution.

### BACKGROUND

This specification relates to identifying and visualizing neuroanatomical tracts in a brain of a biological organism.

A brain may refer to any amount of nervous tissue from a nervous system of a biological organism, and nervous tissue may refer to any tissue that includes neurons (i.e., nerve cells). The biological organism may be, e.g., a worm, a fly, a mouse, a cat, or a human. Pairs of neurons in the brain be connected by synapses. A synapse refers to a structure that enables a neuron to transmit a signal, e.g., an electrical or chemical signal, to another neuron.

### SUMMARY

This specification describes a system implemented as computer programs on one or more computers in one or more locations for identifying and visualizing neuroanatomical tracts in the brain of a biological organism. A neuroanatomical tract, specified by a “seed” neuron, refers to a set of neurons in the brain having the property that each neuron in the set is connected by a respective sequence of one or more synaptic connections to the seed neuron. As used throughout this specification, a seed neuron may be understood to refer, e.g., to a neuron that is selected by a user to specify a neuroanatomical tract.

According to a first aspect there is provided a method performed by one or more data processing apparatus, the method comprising: presenting, to a user and through a display, a representation of a synaptic connectivity graph representing synaptic connectivity between neurons in a brain of a biological organism; receiving, from the user, data specifying a seed neuron in the brain; identifying a neuroanatomical tract corresponding to the seed neuron in the brain, comprising identifying a plurality of neurons in the brain that are each connected to the seed neuron by a respective sequence of one or more synaptic connections; and presenting, to the user and through the display, a geometric representation of at least a portion of the brain of the biological organism that visually distinguishes the neuroanatomical tract corresponding to the seed neuron at neuronal resolution.

In some implementations, the synaptic connectivity graph is defined by a plurality of nodes and edges, wherein each edge connects a pair of nodes, each node corresponds to a respective neuron in the brain of the biological organism, and each edge connecting a pair of nodes in the synaptic connectivity graph corresponds to a synaptic connection between a pair of neurons in the brain of the biological organism.

In some implementations, presenting, to the user and through the display, the representation of the synaptic connectivity graph comprises: presenting, to the user and through the display, a representation of an adjacency matrix corresponding to the synaptic connectivity graph.

In some implementations, identifying the neuroanatomical tract corresponding to the seed neuron in the brain comprises identifying a plurality of neurons in a target region of the brain that are each connected to the seed neuron by a respective sequence of one or more synaptic connections.

In some implementations, the method further includes receiving, from the user, data specifying the target region of the brain.

In some implementations, receiving, from the user, data specifying the target region of the brain comprises: receiving, from the user, data identifying a region of an adjacency matrix representing the synaptic connectivity graph; wherein the target region of the brain comprises each neuron that is connected by a synapse that is specified in the region of the adjacency matrix.

In some implementations, receiving, from the user, data specifying the target region of the brain comprises: receiving, from the user, data specifying a maximum path length; wherein the target region of the brain comprises each neuron that is connected to the seed neuron by a respective sequence of synaptic connections having a length that is at most the maximum path length.

In some implementations, the geometric representation of at least the portion of the brain of the biological organism that visually distinguishes the neuroanatomical tract corresponding to the seed neuron at neuronal resolution comprises a three-dimensional spatial representation of the neurons included in the neuroanatomical tract.

In some implementations, the method further includes obtaining data defining the synaptic connectivity graph, comprising: obtaining a neuronal resolution image of at least a portion of the brain of the biological organism; and processing the image to identify: (i) a plurality of neurons in the brain, and (ii) a plurality of synaptic connections between pairs of neurons in the brain.

In some implementations, the neuronal resolution image of the brain of the biological organism is generated using electron microscopy techniques.

According to a second aspect there is provided a system comprising one or more computers and one or more storage devices communicatively coupled to the one or more computers, wherein the one or more storage devices store instructions that, when executed by the one or more computers, cause the one or more computers to perform operations comprising the method of the first aspect.

According to a third aspect there are provided one or more non-transitory computer storage media storing instructions that when executed by one or more computers cause the one or more computers to perform operations comprising the method of the first aspect.

Particular embodiments of the subject matter described in this specification can be implemented so as to realize one or more of the following advantages.

The system described in this specification enables a user to rapidly identify and visualize neuroanatomical tracts in a brain using a synaptic connectivity graph representing synaptic connectivity in the brain. In particular, the system enables the user to specify a “seed” neuron of interest by viewing a representation of the synaptic connectivity graph, automatically “traces” (i.e., identifies) a corresponding neuroanatomical tract corresponding to the seed neuron, and presents a visualization of the neuroanatomical tract to the user. In this manner, the system enables exploration and visualization of neuroanatomical tracts to be performed more rapidly and effectively than by previous methods, e.g., where an expert person would manually trace neuroanatomical tracts one neuron at a time by examining a microscopic image of the brain. Neuroanatomical visualizations generated by the system can be used in any of a variety of applications, e.g., to guide neurosurgical procedures or to qualitatively characterize changes in the brain over time, as will be described in more detail below.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example data flow for a user of the tracing system to identify and visualize neuroanatomical tracts in a brain.

FIG. 2 shows an example neuroanatomical tract tracing system.

FIG. 3 is a flow diagram of an example process for generating a neuronal resolution geometric representation of a neuroanatomical tract.

Like reference numbers and designations in the various drawings indicate like elements.

### DETAILED DESCRIPTION

This specification describes a neuroanatomical tract tracing system that can be used to identify and visualize neuroanatomical tracts in the brain of a biological organism.

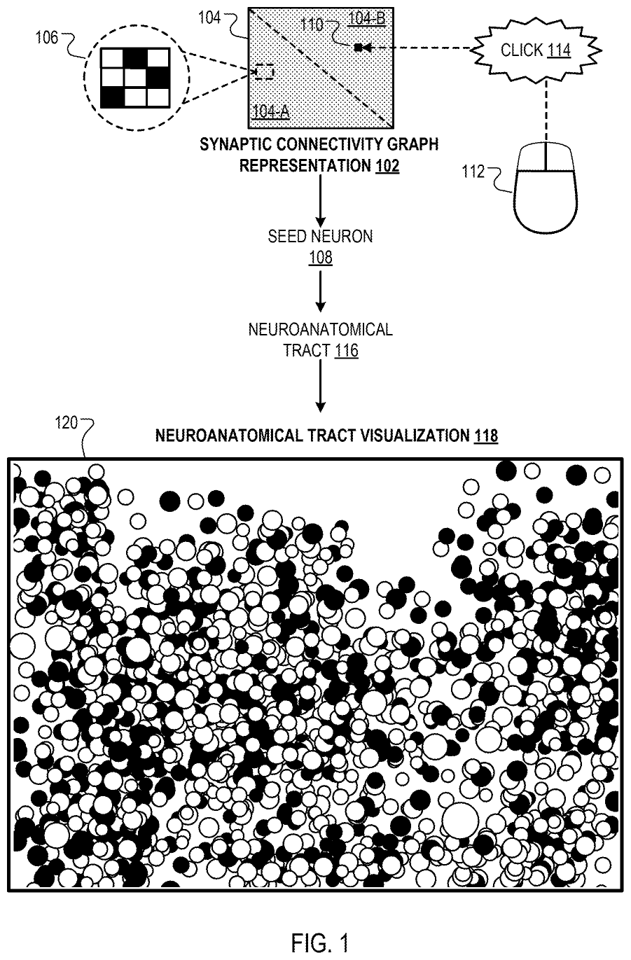

FIG. 1 shows an example data flow for a user of the tracing system to identify and visualize neuroanatomical tracts in a brain.

To assist the user in selecting a seed neuron that specifies the neuroanatomical tract, the tracing system presents a representation of a synaptic connectivity graph 102 that characterizes synaptic connectivity in the brain to the user, e.g., through a display of a user device.

Generally, the synaptic connectivity graph is a graph defined by a set of nodes and edges, where each edge connects a pair of nodes. Each node in the synaptic connectivity graph corresponds to a respective neuron in the brain, and two nodes in the graph are connected if the corresponding neurons in the brain share a synaptic connection. Example techniques for generating the synaptic connectivity graph are described in more detail below.

The tracing system may present any appropriate representation of the synaptic connectivity graph to the user, i.e., through the display of the user device. For example, the tracing system may present an adjacency matrix representing the synaptic connectivity graph to the user. The adjacency matrix may be defined by a two-dimensional array of numerical values with a number of rows and columns equal to the number of nodes in the synaptic connectivity graph. The component of the array at position (i,j) may have a first value (e.g., “1”) if the synaptic connectivity graph includes an edge pointing from node i to node j, and a second, different value (e.g., “0”) otherwise.

The tracing system may present the adjacency matrix on the display of the user device, e.g., as a square grid of cells 104 with a respective cell corresponding to each position in the adjacency matrix. The color of a cell may be specified by the numerical value at the corresponding position in the adjacency matrix. For example, cells corresponding to the value 1 (e.g., indicating the presence of an edge between two nodes) may be colored black, while cells corresponding to the value 0 (e.g., indicating the lack of an edge between two nodes) may be colored white.

In implementations where the synaptic connectivity graph is an undirected graph (i.e., where the edges are not associated with directions), the adjacency matrix may be a symmetric matrix and a representation of the adjacency matrix as a square grid of cells 104 may therefore encode redundant information. To avoid encoding redundant information, the tracing system may represent the adjacency matrix on the display of the user as a triangular grid of cells, e.g., corresponding to the lower-left triangular portion 104-A or the upper-right triangular portion 104-B of the square grid of cells 104.

Generally, the brain may contain large numbers of neurons, e.g., hundreds of thousands or more, and a user interface available to the user through the user device may enable the user to examine the representation of the synaptic connectivity graph 102 at various magnifications. In particular, the user interface may enable the user to “zoom-in” on (i.e., magnify) portions of the representation of the synaptic connectivity graph 102 to more readily distinguish synaptic connections between individual neurons, e.g., as illustrated by the magnified portion 106 of the adjacency matrix 104.

By viewing the representation of the synaptic connectivity graph 102, the user may identify a seed neuron 108 of interest, and thereafter provide an input to the user device that specifies the seed neuron 108. For example, the user may provide an input by selecting a cell 110 of the adjacency matrix 104 representing the synaptic connectivity graph, thereby specifying either one of the pair of neurons corresponding to the cell 110 of the adjacency matrix 104 as the seed neuron 108. More specifically, selecting the cell at position (i,j) in the adjacency matrix 104 may specify either one of the neurons indexed by indices i and j as the seed neuron 108.

The user may interact with the user device to specify the seed neuron 108 in any of a variety of ways. For example, the user may specify the seed neuron 108 by using a mouse 112 to “click” 114 (or otherwise select) a cell in the adjacency matrix representing the synaptic connectivity graph. As another example, the user may specify the seed neuron 108 by using a keyboard to input the numerical index corresponding to the seed neuron 108.

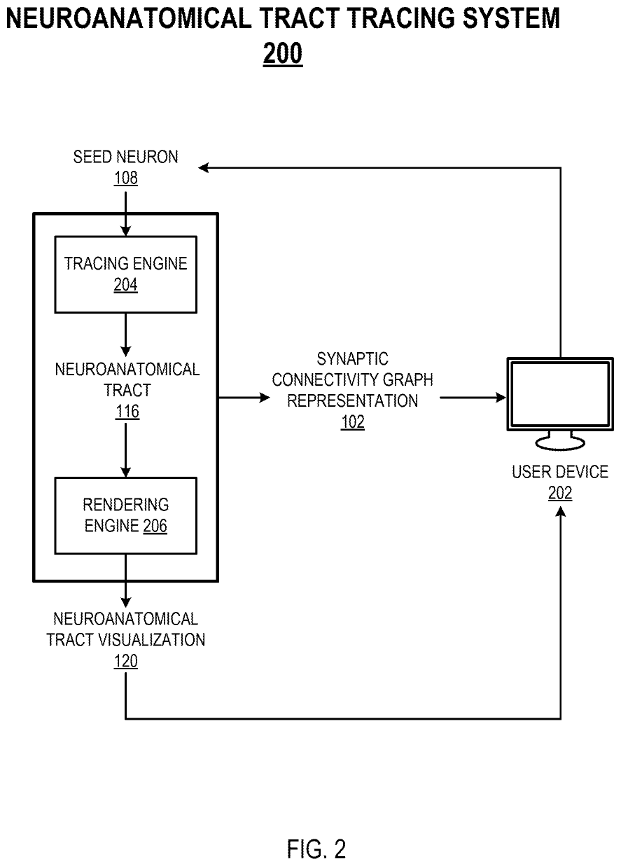

After the user selects the seed neuron 108, the tracing system identifies a neuroanatomical tract 116 that is specified by the seed neuron 108. The neuroanatomical tract 116 is defined by a set of neurons in the brain having the property that each neuron in the set is connected by a respective sequence of one or more synaptic connections to the seed neuron 108. Generally, the neuroanatomical tract 116 is defined to include the seed neuron 108 itself. Example techniques for identifying a neuroanatomical tract 116 corresponding to a seed neuron 108 are described in more detail with reference to FIG. 2.

The tracing system then presents a visualization 120 of the neuroanatomical tract 116 to the user through the display of the user device. More specifically, the tracing system may present a visualization 120 of the neuroanatomical tract 116 that is a neuronal resolution geometric representation of the brain that visually distinguishes the neuroanatomical tract 116. The visualization 120 is referred to as having “neuronal resolution” because, when viewed at an appropriate magnification, the visualization 120 individually depicts each respective neuron in the neuroanatomical tract 116. The visualization 120 is referred to as being a “geometric representation” because it illustrates the neurons in three-dimensional (3-D) space, such that the respective spatial positions of the neurons in the visualization 120 are consistent with the respective spatial positions of the neurons in the brain.

In the example neuroanatomical tract visualization 120, neurons are depicted as circles, where shaded circles represent neurons that are included in the neuroanatomical tract 116, and unshaded circles represent neurons that are not included in the neuroanatomical tract 116.

The example visualization 120 is provided for illustrative purposes only, and other visualizations are possible. For example, the visualization may be formatted to depict only the neurons that are included in the neuroanatomical tract 116, in contrast to the example visualization 120, where neurons that are not included in the neuroanatomical tract 116 are also depicted. In another example, the visualization may depict the respective synaptic connections between the neurons in the neuroanatomical tract, in contrast to the visualization 120, where the synaptic connections are not depicted.

In some cases, the user interface may enable the user to interact with the visualization 120. For example, the user interface may enable the user to rotate the visualization 120, increase or reduce the magnification at which the visualization 120 is depicted, and toggle options such as whether neurons outside the neuroanatomical tract 116 are depicted in the visualization 120.

Optionally, the tracing system may enable the user to specify multiple seed neurons 108, identify a respective neuroanatomical tract 116 corresponding to each seed neuron 108, and present a visualization that jointly or separately depicts the respective neuroanatomical tract 116 corresponding to each seed neuron 108. In one example, the tracing system may illustrate the respective neuroanatomical tract 116 corresponding to each of multiple seed neurons 108 using a respective color.

The representation of the synaptic connectivity graph 102 and the neuroanatomical tract visualization 120 may be presented to the user in any of a variety of ways. In one example, the neuroanatomical tract visualization 120 may be presented in the center of the display, and the synaptic connectivity graph may be presented in a corner of the display. In some cases, additional statistical data associated with the neuroanatomical tract may also be presented, e.g., the number of neurons in the neuroanatomical tract, the average number of synapses connected to neurons in the neuroanatomical tract, or both.

Neuroanatomical tract visualizations 118 generated by the tracing system can be used for any of a variety of purposes. A few examples are describe in more detail next.

In one example, the neuroanatomical tract visualizations 118 may be used to guide a neurosurgical procedure. In particular, a physician may consult the visualization 120 to assess the potential consequences of damaging certain parts of the brain during a procedure. For example, the physician may determine, based on the visualization, that certain neurons are included in a neuroanatomical tract that passes through a portion of the brain associated with certain functions, e.g., motor control or language processing functions, and thereafter refrain from damaging those neurons.

In another example, the neuroanatomical tract visualizations 118 may be used to qualitatively assess and visualize changes in the brain over time, e.g., as a result of administering a drug or treatment. For example, a respective synaptic connectivity graph characterizing the brain of a patient receiving a treatment may be generated at each of multiple time points (e.g., every 30 days), and neuroanatomical tract visualizations 118 may be generated from each synaptic connectivity graph to visualize changes in the brain.

FIG. 2 shows an example neuroanatomical tract tracing system 200. The neuroanatomical tract tracing system 200 is an example of a system implemented as computer programs on one or more computers in one or more locations in which the systems, components, and techniques described below are implemented.

The tracing system 200 provides a representation of a synaptic connectivity graph 102 representing synaptic connectivity in a brain for presentation on a user device 202. For example, the tracing system 200 may provide a representation of an adjacency matrix specifying the synaptic connectivity graph for presentation on the user device 202, as described above.

The synaptic connectivity graph may be generated in any of a variety of ways. For example, a graphing system may generate the synaptic connectivity graph by obtaining a synaptic/neuronal resolution image of the brain (a “brain image”) and processing the brain image to identify the neurons and synapses depicted in the brain image. The graphing system may then generate the synaptic connectivity graph by instantiating a respective node corresponding to each neuron in the brain image, and instantiating an edge between any pair of nodes that correspond to a pair of neurons sharing a synaptic connection.

In addition to identifying synapses in the brain image, the graphing system may further determine the direction of each synapse using any appropriate technique. The “direction” of a synapse between two neurons refers to the direction of information flow between the two neurons, e.g., if a first neuron uses a synapse to transmit signals to a second neuron, then the direction of the synapse would point from the first neuron to the second neuron. Example techniques for determining the directions of synapses connecting pairs of neurons are described with reference to: C. Seguin, A. Razi, and A. Zalesky: “Inferring neural signalling directionality from undirected structure connectomes,” Nature Communications 10, 4289 (2019), doi:10.1038/s41467-019-12201-w.

Where the graphing system determines the directions of the synapses in the brain image, the graphing system may associate each edge in the synaptic connectivity graph with the direction of the corresponding synapse. That is, the synaptic connectivity graph may be a directed graph. In other implementations, the synaptic connectivity graph may be an undirected graph, i.e., where the edges in the graph are not associated with a direction.

An example process for generating a synaptic connectivity graph from a brain image is described in more detail with reference to U.S. patent application Ser. No. 16/776,108, which is hereby incorporated by reference. For example, the graphing system described with reference to FIG. 2 of U.S. patent application Ser. No. 16/776,108 provides one example of a system for generating a synaptic connectivity graph from a brain image.

The user device may be any appropriate device that includes a visual display and can receive user inputs. In some cases, the tracing system 200 may be implemented locally on the user device 202, while in other cases, the tracing system 200 may be implemented remotely from the user device 202 (e.g., in a cloud environment) and communicate with the user device over a network, e.g., the Internet. Communicating with the user device refers to transmitting data to the user device and receiving data from the user device.

A user of the user device 202 may, in response to viewing the representation of the synaptic connectivity graph 102, identify a seed neuron 108 of interest and provide an input to the user device 202 that specifies the seed neuron 108. For example, the user may provide an input by selecting a position in the adjacency matrix representing the synaptic connectivity graph, thereby specifying one or both of the neurons corresponding to the position as seed neurons 108, as described above.

The tracing system 200 receives data specifying the seed neuron 108 and generates a visualization 120 of a neuroanatomical tract 116 corresponding to the seed neuron 108 using a tracing engine 204 and a rendering engine 206, which will each be described in more detail next.

The tracing engine 204 is configured to generate data defining the neuroanatomical tract 116 corresponding to the seed neuron 108. The neuroanatomical tract 116 is defined by a set of neurons in the brain having the property that each neuron in the set is connected to the seed neuron 108 by a respective sequence of one or more synaptic connections. The neuroanatomical tract 116 is generally defined to also include the seed neuron 108 itself. To identify the neuroanatomical tract 116, the tracing engine 204 may identify each node in the synaptic connectivity graph that is connected (i.e., by a sequence of one or more edges) to the node corresponding to the seed neuron (referred to herein as the “seed node”). For example, the tracing engine 204 may perform a breadth-first search of the synaptic connectivity graph starting from the seed node. The tracing engine 204 may then identify each neuron that corresponds to a node that is connected to the seed node in the synaptic connectivity graph as being included in the neuroanatomical tract 116.

In some implementations, in addition to specifying a seed neuron 108, the user provides an input specifying a “target region” of the brain, i.e., a set of neurons in the brain that are eligible to be included in the neuroanatomical tract 116 corresponding to the seed neuron 108. In these implementations, the tracing engine 204 restricts the neuroanatomical tract 116 to only those neurons that are included in the target region of the brain. A few examples of user inputs that specify a target region of the brain are described in more detail next.

In one example, a user input may specify a target region of the brain by specifying a positive integer value defining a “maximum path length” parameter. In this example, the target region of the brain includes only those neurons that are connected to the seed neuron 108 by at least one sequence of synaptic connections having a length that is at most the maximum path length. The “length” of a sequence of synaptic connections refers to the number of synaptic connections in the sequence.

In another example, a user input may specify a target region of the brain by identifying a region of an adjacency matrix representing the synaptic connectivity graph, e.g., a sub-matrix of the adjacency matrix. In this example, the target region of the brain includes only those neurons that are connected by synapses that are specified in the identified region of the adjacency matrix.

In another example, a user input may specify a target region of the brain by specifying a particular function performed by the brain, e.g., a motor control function or a language processing function. In this example, the target region of the brain includes only those neurons that are inside a predefined spatial region of the brain that is associated with the particular function.

Certain anatomical tracts may include large numbers of neurons, e.g., thousands of neurons or more. Restricting the neuroanatomical tract 116 corresponding to the seed neuron 108 to a target region of the brain may reduce the number of neurons included in the neuroanatomical tract 116, which may reduce the computational resources required to identify the neuroanatomical tract 116 and to generate the visualization 120.

The rendering engine 206 is configured to generate the visualization 120 of the neuroanatomical tract 116. To generate the visualization 120, the rendering engine 206 may associate each neuron in the neuroanatomical tract with data characterizing the spatial position of the neuron in the brain, e.g., coordinates in a 3-D coordinate system, e.g., a Cartesian coordinate system. The spatial positions of neurons in the brain may be derived from the synaptic resolution brain image that was used to generate the synaptic connectivity graph, as described above.

After associating each neuron in the neuroanatomical tract 116 with corresponding spatial position data, the rendering engine 206 generates a visualization 120 of the neuroanatomical tract 116 that depicts each neuron at the corresponding spatial position. Optionally, the visualization 120 may depict additional features of the neuroanatomical tract 116, e.g., the synapses connecting the neurons in the neuroanatomical tract 116, and other neurons that are outside the neuroanatomical tract. An example visualization 120 of a neuroanatomical tract is illustrated in FIG. 1.

After generating the visualization 120, the tracing engine 204 provides the visualization 120 of the neuroanatomical tract 116 corresponding to the seed neuron 108 for presentation on the display of the user device 202.

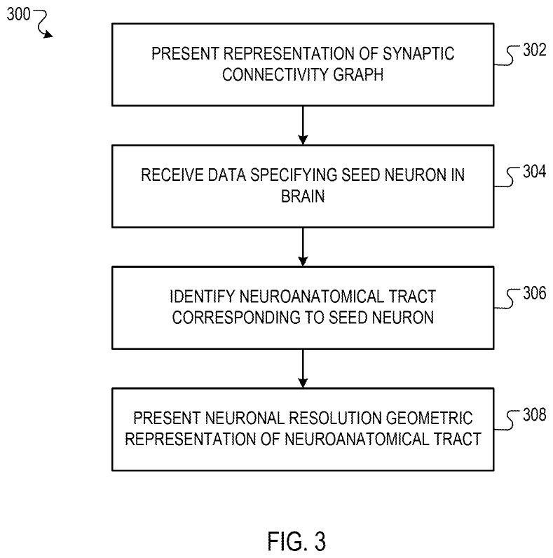

FIG. 3 is a flow diagram of an example process 300 for generating a neuronal resolution geometric representation of a neuroanatomical tract. For convenience, the process 300 will be described as being performed by a system of one or more computers located in one or more locations. For example, a neuroanatomical tract tracing system, e.g., the neuroanatomical tract tracing system 200 of FIG. 2, appropriately programmed in accordance with this specification, can perform the process 300.

The system presents, to a user and through a display, a representation of a synaptic connectivity graph representing synaptic connectivity between neurons in a brain of a biological organism (302).

The system receives, from the user, data specifying a seed neuron in the brain (304).

The system identifies a neuroanatomical tract corresponding to the seed neuron in the brain, including identifying a set of neurons in the brain that are each connected to the seed neuron by a respective sequence of one or more synaptic connections (306).

The system presents, to the user and through the display, a neuronal resolution geometric representation of at least a portion of the brain of the biological organism that visually distinguishes the neuroanatomical tract corresponding to the seed neuron in the brain (308).

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, a Microsoft Cognitive Toolkit framework, an Apache Singa framework, or an Apache MXNet framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method performed by one or more data processing apparatus, the method comprising:
presenting, to a user and through a display, a representation of a synaptic connectivity graph representing synaptic connectivity between neurons in a brain of a biological organism;
receiving, from the user, data specifying a seed neuron in the brain;
identifying a neuroanatomical tract corresponding to the seed neuron in the brain, comprising identifying a plurality of neurons in the brain that are each connected to the seed neuron by a respective sequence of one or more synaptic connections; and
presenting, to the user and through the display, a geometric representation of at least a portion of the brain of the biological organism that visually distinguishes the neuroanatomical tract corresponding to the seed neuron at neuronal resolution.

2. The method of claim 1, wherein the synaptic connectivity graph is defined by a plurality of nodes and edges, wherein each edge connects a pair of nodes, each node corresponds to a respective neuron in the brain of the biological organism, and each edge connecting a pair of nodes in the synaptic connectivity graph corresponds to a synaptic connection between a pair of neurons in the brain of the biological organism.

3. The method of claim 2, wherein presenting, to the user and through the display, the representation of the synaptic connectivity graph comprises:
presenting, to the user and through the display, a representation of an adjacency matrix corresponding to the synaptic connectivity graph.

4. The method of claim 1, wherein identifying the neuroanatomical tract corresponding to the seed neuron in the brain comprises identifying a plurality of neurons in a target region of the brain that are each connected to the seed neuron by a respective sequence of one or more synaptic connections.

5. The method of claim 4, further comprising receiving, from the user, data specifying the target region of the brain.

6. The method of claim 5, wherein receiving, from the user, data specifying the target region of the brain comprises:
receiving, from the user, data identifying a region of an adjacency matrix representing the synaptic connectivity graph;
wherein the target region of the brain comprises each neuron that is connected by a synapse that is specified in the region of the adjacency matrix.

7. The method of claim 5, wherein receiving, from the user, data specifying the target region of the brain comprises:
receiving, from the user, data specifying a maximum path length;
wherein the target region of the brain comprises each neuron that is connected to the seed neuron by a respective sequence of synaptic connections having a length that is at most the maximum path length.

8. The method of claim 1, wherein the geometric representation of at least the portion of the brain of the biological organism that visually distinguishes the neuroanatomical tract corresponding to the seed neuron at neuronal resolution comprises a three-dimensional spatial representation of the neurons included in the neuroanatomical tract.

9. The method of claim 1, further comprising obtaining data defining the synaptic connectivity graph, comprising:
obtaining a neuronal resolution image of at least a portion of the brain of the biological organism; and
processing the image to identify: (i) a plurality of neurons in the brain, and (ii) a plurality of synaptic connections between pairs of neurons in the brain.

10. The method of claim 9, wherein the neuronal resolution image of the brain of the biological organism is generated using electron microscopy techniques.

11. A system comprising:
one or more computers; and
one or more storage devices communicatively coupled to the one or more computers, wherein the one or more storage devices store instructions that, when executed by the one or more computers, cause the one or more computers to perform operations comprising:
presenting, to a user and through a display, a representation of a synaptic connectivity graph representing synaptic connectivity between neurons in a brain of a biological organism;
receiving, from the user, data specifying a seed neuron in the brain;
identifying a neuroanatomical tract corresponding to the seed neuron in the brain, comprising identifying a plurality of neurons in the brain that are each connected to the seed neuron by a respective sequence of one or more synaptic connections; and
presenting, to the user and through the display, a geometric representation of at least a portion of the brain of the biological organism that visually distinguishes the neuroanatomical tract corresponding to the seed neuron at neuronal resolution.

12. The system of claim 11, wherein the synaptic connectivity graph is defined by a plurality of nodes and edges, wherein each edge connects a pair of nodes, each node corresponds to a respective neuron in the brain of the biological organism, and each edge connecting a pair of nodes in the synaptic connectivity graph corresponds to a synaptic connection between a pair of neurons in the brain of the biological organism.

13. The system of claim 12, wherein presenting, to the user and through the display, the representation of the synaptic connectivity graph comprises:
presenting, to the user and through the display, a representation of an adjacency matrix corresponding to the synaptic connectivity graph.

14. The system of claim 11, wherein identifying the neuroanatomical tract corresponding to the seed neuron in the brain comprises identifying a plurality of neurons in a target region of the brain that are each connected to the seed neuron by a respective sequence of one or more synaptic connections.

15. The system of claim 14, further comprising receiving, from the user, data specifying the target region of the brain.

16. The system of claim 15, wherein receiving, from the user, data specifying the target region of the brain comprises:
receiving, from the user, data identifying a region of an adjacency matrix representing the synaptic connectivity graph;
wherein the target region of the brain comprises each neuron that is connected by a synapse that is specified in the region of the adjacency matrix.

17. The system of claim 15, wherein receiving, from the user, data specifying the target region of the brain comprises:
receiving, from the user, data specifying a maximum path length;
wherein the target region of the brain comprises each neuron that is connected to the seed neuron by a respective sequence of synaptic connections having a length that is at most the maximum path length.

18. The system of claim 11, wherein the geometric representation of at least the portion of the brain of the biological organism that visually distinguishes the neuroanatomical tract corresponding to the seed neuron at neuronal resolution comprises a three-dimensional spatial representation of the neurons included in the neuroanatomical tract.

19. The system of claim 11, further comprising obtaining data defining the synaptic connectivity graph, comprising:
obtaining a neuronal resolution image of at least a portion of the brain of the biological organism; and
processing the image to identify: (i) a plurality of neurons in the brain, and (ii) a plurality of synaptic connections between pairs of neurons in the brain.

20. One or more non-transitory computer storage media storing instructions that when executed by one or more computers cause the one or more computers to perform operations comprising:
presenting, to a user and through a display, a representation of a synaptic connectivity graph representing synaptic connectivity between neurons in a brain of a biological organism;
receiving, from the user, data specifying a seed neuron in the brain;
identifying a neuroanatomical tract corresponding to the seed neuron in the brain, comprising identifying a plurality of neurons in the brain that are each connected to the seed neuron by a respective sequence of one or more synaptic connections; and
presenting, to the user and through the display, a geometric representation of at least a portion of the brain of the biological organism that visually distinguishes the neuroanatomical tract corresponding to the seed neuron at neuronal resolution.
